(19) United States
(12) Patent Application Publication
(10) Pub. No.: US 2006/0205089 A1
(43) Pub. Date: Sep. 14, 2006

(54) Mapping discontinuous antibody or aptamer epitopes for protein structure determination and other applications

(75) Inventors: Edward A. Dratz, Bozeman, MT (US); Brendan M. Mumey, Bozeman, MT (US); Algirdas J. Jesaitis, Bozeman, MT (US)

Correspondence Address: DORSEY & WHITNEY LLP, 555 CALIFORNIA STREET, SUITE 1000, SUITE 1000, SAN FRANCISCO, CA, 94104, US

(73) Assignee: MONTANA STATE UNIVERSITY, Bozeman, MT (US)

(21) Appl. No.: 10/552,930

(22) PCT Filed: Apr. 14, 2004
(86) PCT No.: PCT/US04/11905

§ 371 Date: Oct. 14, 2005

Related U.S. Application Data
(60) Provisional application No. 60/462,870, filed on Apr. 14, 2003.

(51) Int. Cl. G01N 33/543

(52) U.S. Cl. 436/518

(57) ABSTRACT

The present invention is directed to methods of mapping antibody or aptamer epitopes for use in a variety of utilities including the elucidation of the tertiary structure of proteins. A method of the invention, antibody imprinting, uses antibodies or aptamers against target proteins and random peptide probe libraries to map the epitopes of antibody binding sites on target proteins.

This application claims the benefit of the priority date of U.S. Ser. No. 60/462,870 filed on Apr. 14, 2003, hereby expressly incorporated by reference.

### GOVERNMENT INTERESTS

This research was supported by the US National Institutes of Health Grants NIH 1R01 GM62547, NIH 1R011 RO1 AI22735, and NIH 1R011R01 AI26711.

### FIELD OF THE INVENTION

The invention relates to the mapping of discontinuous epitopes of antibodies to target proteins for a variety of utilities.

### BACKGROUND OF THE INVENTION

Proteins are nano-machines that are constructed from long chains of amino acids (typically 100-1000 elements) using twenty different amino acids arranged in characteristic sequences. Proteins must be folded into complex 3-D shapes to create the binding pockets and active sites necessary to carry out their myriad of different functions [Branden and Tooze, 1999]. There are at least 30,000 different proteins in human cells [Clayerie, 2001] and each protein has a folded functional structure. Whenever the 3-D folded structure of linear protein sequences can be determined this information has provided important insights into mechanisms of action and may be extremely useful in drug design. Traditional methods of protein structure determination require preparation of large amounts of protein in functional form, which often may not be feasible. Given sufficient protein of interest, conditions are screened to seek 3-D crystals for structure determination by x-ray diffraction, however, obtaining crystals of sufficient quality may not be possible [McPherson, 1999, Michel, 1990]. Alternatively, if the proteins are not too large, are highly water soluble, and meet other criteria, methods of nuclear magnetic resonance can be used for structure determination [Cavanagh et al., 1996]. It is also possible to predict 3-D structures de novo from the sequence of amino acids in the protein, but the available methods for structure prediction are not very accurate unless a 3-D structure of a homologous protein is already known [Baker and SalI, 2001].

A large fraction of protein structures of interest (50% or more) cannot be solved by the traditional approaches discussed above [Edwards et al., 2000, Eisenstein et al., 2000].

Monoclonal antibodies are in widespread use as therapeutics, diagnostics, and research reagents. As therapeutics, antibodies are used to treat a variety of conditions including cancer, autoimmune diseases, and cardiovascular disease. There are currently over ten approved antibody products on the US market, with over a hundred in development. Despite such acceptance and promise, there remains significant need for optimization of the structural and functional properties of antibodies and to better understand the mechanism of antibody interactions with their protein targets.

The physical and chemical properties of antibody therapeutics significantly determine their performance during development, manufacturing, and clinical use. Antibodies may suffer from the stability and solubility issues similar to all proteins. Since fully developed antibody therapeutics require high levels of stability and solubility in order to retain activity through purification, formulation, storage, and administration, there is a need for effective methods to both optimize antibody properties as well as engineer new antibodies to proteins traditionally difficult to raise antibodies against. In addition, there is a need to use antibodies to help elucidate tertiary structure of proteins not obtainable in traditional methods.

### SUMMARY OF THE INVENTION

In accordance with the objects outlined above, the present invention provides methods for mapping epitopes on a target protein. One aspect of the invention provides for a method of mapping surface epitopes on a target protein comprising the steps of providing a solid support comprising an antibody capable of binding to the target protein, contacting the solid support with a library of random peptides under conditions where a set of probe peptides bind to the antibody, eluting the set of probe peptides, determining the amino acid sequence of the members of the set of probe peptides, computationally aligning the probe peptide sequences to the target protein, generating a set of best alignments for each probe peptide sequence, generating a surface-neighbor probability graph from the alignments, determining edge weight of the surface-neighbor probability graph, and constructing at least one surface epitope of the target protein from the surface-neighbor probability graph. In a preferred embodiment, the surface epitope mapped by the method is a discontinuous epitope. In a further preferred embodiment, the computational aligning of the probe peptides to the target protein comprises a branch and bound program. In another preferred embodiment, the library of random peptides is a phage display library. In a further preferred embodiment, the phage display library is enriched.

Another aspect of the invention provides for a method of mapping discontinuous epitopes on a target protein comprising the steps of providing a solid support comprising an antibody capable of binding to the target protein, contacting the solid support with a library of random peptides under conditions where a set of probe peptides bind to the antibody, eluting the set of probe peptides, determining the amino acid sequence of the members of the set of probe peptides, computationally aligning the amino acid sequences of the probe peptide sequences to the target protein, and constructing at least one discontinuous epitope on the target protein. In a preferred embodiment, the computational aligning of the probe peptides to the target protein comprises a branch and bound program. In another preferred embodiment, the library of random peptides is a phage display library. In a further preferred embodiment, the phage display library is enriched.

Another aspect of the invention provides for a method of mapping discontinuous epitopes on a target protein comprising the steps of providing a solid support comprising an antibody capable of binding to the target sequence, contacting the solid support with a library of random peptides under conditions where a set of probe peptides bind to the antibody, eluting the set of probe peptides, determining the amino acid sequence of the members of the set of probe peptides, and determining a consensus epitope sequence, computationally aligning the consensus epitope sequence to the target protein, constructing at least one discontinuous epitope on the protein. In a preferred embodiment, the computational aligning of the probe peptides to the target protein comprises a branch and bound program. In another preferred embodiment, the library of random peptides is a phage display library. In a further preferred embodiment, the phage display library is enriched.

Another aspect of the invention provides for a method of mapping surface epitopes on a target protein comprising the steps of providing a solid support comprising an aptamer capable of binding to the target protein, contacting the solid support with a library of random peptides under conditions where a set of probe peptides bind to the antibody, eluting the set of probe peptides, determining the amino acid sequence of the members of the set of probe peptides, computationally aligning the probe peptide sequences to the target protein, generating a set of best alignments for each probe peptide sequence, generating a surface-neighbor probability graph from the alignments, determining edge weight of the surface-neighbor probability graph, and constructing at least one surface epitope of the target protein from the surface-neighbor probability graph.

Yet another aspect of the invention provides for a method of mapping discontinuous epitopes on a target protein comprising the steps of providing a solid support comprising an aptamer capable of binding to the target sequence, contacting the solid support with a library of random peptides under conditions where a set of probe peptides bind to the antibody, eluting the set of probe peptides, determining the amino acid sequence of the members of the set of probe peptides, determining a consensus epitope sequence, computationally aligning the consensus epitope sequence to the target protein, and constructing at least one discontinuous epitope on the protein.

Yet another aspect of the invention provides for a method of mapping protein-protein binding sites comprising the steps of providing a solid support comprising a first protein, contacting the solid support with a second protein under conditions where the first protein binds to the second protein, contacting the solid support with a library of random peptides under conditions where a set of probe peptides compete with the second protein for binding to the first protein, eluting the set of probe peptides, determining the amino acid sequences of the members of the set of probe peptides, computationally aligning the amino acid sequences of the members of the set of probe peptides to the target protein, and constructing the binding site on the protein.

### BRIEF DESCRIPTION OF THE DRAWINGS

FIG. 1: An example of an amino acid substitution scoring matrix used in FINDMAP. This particular matrix is based on the probability of amino acid substitutions on surface-exposed residues of proteins. The Bordo and Argos [Bordo and Argos, 1991] substitution matrix was modified so that Gly/Pro substitutions score 0.50, Arg/His, Lys/His, and Gly/Ser substitutions score 0.25. Unaligned probe positions were charged a penalty of −1. The gap penalty discussed in the text was levied against gaps in the target protein sequence that were not aligned with probe residues. Different substitution matrices can be substituted in FINDMAP and matrices can be optimized by the use of known calibration leaming sets of protein-antibody complexes with known 3D structures.

FIG. 2: Mapping of the anti-actin antibody epitope VPHPTWMR onto the surface of actin manually and by FINDMAP. Mapped residues are indicated by arrows and proceed in a counterclockwise direction beginning with Val 129 for the FINDMAP results, based on the probe peptide sequence from N to C-terminal. The manual and FINDMAP mappings differ only in their alignment of Thr 358 where FINDMAP tends to pick Thr 103. The independent manual mapping required knowledge of the actin x-ray structure. The top-scoring FINDMAP alignment having the best match is shown above the actin sequence (#'s indicate residues known to be folded away from the aqueous surface of actin; these regions were excluded in the manual mapping).

FIG. 3: An example of a FINDMAP epitope gap penalty parameter sensitivity test. For the gap distance penalty function d(n)=min(a·n,b), various combinations of a and b where tested on mapping the probe epitope VPHPTWMR to the actin sequence (see FIG. 2). These alignments were ranked in three categories, based on how closely they agreed with the published manual mapping to the known 3-D structure of actin [Jesaitis et al., 1999]. The diamond-shaped points in the figure indicate parameter combinations where FINDMAP found the published mapping to within one residue position as one of the top-scoring alignments, the parameter combinations used to yield the square points missed two or three residues and the triangular region more than three residues.

FIGS. 4A and 4B: Mapping of the 4B4 antibody epitope on rhodopsin. Panel A shows the epitope of the 4B4 antibody mapped on the cytoplasmic surface of a model of the 3-D structure of dark-adapted rhodopsin. This region is not resolved In the x-ray crystal structure, as explained in the text. The 4B4 consensus probe EQQVSATAQ was best aligned, using FINDMAP, to the rhodopsin residues EQQASATTQ. Mapped epitope residues are indicated by arrows. Different cytoplasmic regions of the protein are indicated by shaded residues. Panel B shows the proposed reorientation of residues 235-244 of the C-3 loop of rhodopsin, with A235 moved next to S240, based on the best-scoring FINDMAP alignment, as discussed in the text.

FIGS. 5A-5D: Surface Neighbor Graphs and Corresponding Protein: Surface neighbor graphs (panels A and C) contain numbers in rectangular boxes that indicate the residue sequence numbers mapped in the proteins considered. The edge weights in the graphs are shaded according to their strength. Panel A: Actin surface neighbor graph constructed as described in the text from the collection of top-scoring alignments for a set of 90 experimental probe epitope mimetic peptides found for a polyclonal antibody against actin [Jesaitis et al., 1999]. Panel B: The physical location of the residues In the crystal structure of actin (PDB: 1ATN) mapped in panel A. Panel C: Surface neighbor graph of lysozyme constructed as described in the text. Note: Gap cost parameters were optimized separately for lysozyme, a=0.3,b=1.0 were found to yield the best alignments. Panel D: Known Hyhel-10 antibody epitope on lysozyme determined experimentally from the xray crystal structure of the complex (PDB: 1C08).

### DETAILED DESCRIPTION OF THE INVENTION

The present invention is directed to the mapping of discontinuous antibody or aptamer epitopes for a variety of reasons, including the elucidation of structural information on target proteins as well as for the development of improved antibodies. Accordingly, the present invention Is directed to a new method, termed “antibody imprinting”, to provide structural information on difficult target protein cases that appear refractory to traditional approaches [Burritt et al., 1998, Jesaitis et al., 1999, Bailey et al., 2003]. The antibody imprint method makes use of information carried in the structures of antibodies against proteins of interest to reveal the 3-D folding of target proteins [Burritt et al., 1998, Jesaitis et al., 1999, Demangel et al., 2000, Heiskanen et al., 1999, Bailey et al., 2001, 2003]. Antibodies tend to be highly specific for the protein structures that they recognize [Janeway and Travers, 1996]. Antibodies can either recognize continuous or discontinuous epitopes. Discontinuous epitopes provide the most useful structural Information in antibody imprinting, because they can reveal distant segments of primary sequence that are in close spatial proximity on the native, folded protein. Evidence to date indicates that most antibodies recognize discontinuous epitopes on protein surfaces [Padlan, 1996]. Studies of a substantial number of antibody-protein complexes with known x-ray structures indicate that these complexes form in a lock and key manner, with little or no structural change induced by complex formation [Conte et al., 1999]. Fortunately, relatively few long-distance constraints are needed to reveal the global folding of proteins [Clore et al., 1993, Dandekar and Argos, 1997]. In addition, the spatial proximity of different regions of proteins can change during function and antibody imprinting has the potential to reveal these structural changes, if appropriate antibodies can be found that recognize the different structural shapes [Bailey et al., 2001, 2003].

Briefly, the antibody Imprinting method Is carried out by first immobilizing antibodies (against a target protein of interest) on a solid support such as beads or in plastic wells. The immobilized antibodies are exposed to random peptide libraries to capture library members that bind to the antibodies. These “probe” peptides can then be computationally aligned or mapped onto the target protein to elucidate the structure of the protein and/or epitope. Essentially, a “positive” (the target protein) is used to make a “negative” (e.g. the antibody) which is used to recreate a new “positive” (the discontinuous epitope), using a computational approach, for example a branch and bound algorithm or program. The method is adaptable to the substitution of single stranded nucleic acid aptamers instead of antibodies. In some cases aptamers can be selected to have higher affinities and specificities than antibodies.

In addition, while the discussion below focuses on alignment of antibodies and target proteins, it should be noted that this technique can be used to map protein-protein interactions. In one embodiment, this may be done using competitive assays. For example, a first protein can be affixed to the solid support, and the known binding protein bound to it. The library of random peptides is passed over the solid support, and peptides with affinities higher than the known binding protein will displace the known protein. These peptides are then eluted and analyzed as discussed herein. Alternatively, general binding domains of the bound protein can be elucidated by utilizing the library and then mapping the surface to determine the structure of the binding domain. Alternatively, if antibodies (including aptamers) are made to the protein-protein contact regions, the system described herein can be used to map that interaction.

The random peptide libraries fall into two general classes; either the peptides are “displayed” in some fusion, for example as fusion peptides in phage display systems, displayed on ribosomes, bound to marker beads, displayed using presentation structures that allow the peptide to be held in a conformationally constrained manner or they may be free in solution.

In a preferred embodiment, the peptide libraries are carried on bacteriophage (referred to as “phage display” of the library), as is reviewed in the following reference [Barbas et al., 2001]. Each phage has a different peptide expressed on the surface of one of its coat proteins and there are typically 5-10⁹ [Burritt et al., 1996] and even up to 10¹² different peptide sequences in each library [Sidhu et al., 2000]. These probe libraries contain linear or constrained (for example, “displayed”) peptides (including but not limited to circular topology, where the two ends of the probe are chemically linked with a disulfide bond). Peptide sequences that do not stick to the antibody are washed off the immobilized antibodies and the tightly binding phage are eluted under harsher conditions. Peptides with different binding affinities may be elucidated by differented elution conditions. These phage are then diluted and grown as clones that arise from individual phage particles. Each of the phage clones carry the DNA sequence that codes for the peptide sequence that has been selected. The DNA sequence of selected clones is determined via any nucleic acid sequence method and the amino acid sequence of the peptide determined from the DNA sequence. In a preferred embodiment, the DNA regions of the selected clones are amplified by PCR with optionally fluorescent terminators and sequenced in a standard automated DNA sequencer.

In a preferred embodiment, the phages that bind to the antibody are multiplied by growth in suitable bacteria and reiteratively exposed again to the immobilized antibody. These cycles of binding and enrichment of members of the random peptide library are usually repeated 1-5 times to select the phage or with the highest affinity to the antibody. As above, these enriched phage are then identified.

In this way, the sequence for each epitope-mimetic peptide is discovered. These individual sequences are often highly conserved and 25-100 independent peptide sequences together describe a consensus sequence, herein called the “consensus epitope” of the antibody. In some cases, a consensus epitope can be generated; in others overlapping epitope-mimetic pepbdes are used.

Alternatively, random peptide libraries may be made by expression in any number of systems, including expression in host cells, display on ribosomes, display on viruses (particularly retroviruses), or by chemical synthesis on beads which can be retained on the beads or cleaved and used in solution. In these embodiments, there are preferably rescue and/or amplification sequences (described below) that allow the peptides to be replicated for elucidation.

Accordingly, the present invention is directed to methods of mapping discontinuous epitopes on a target protein. By “epitope” herein in meant that part of the target protein (e.g. the amino acids making up the antigen) which is recognized by (e.g. binds to) the antibody (e.g. antigen receptor) or aptamer. In the case of protein/protein interactions, an “epitope” in this context means a binding site-mimetic. By “discontinuous epitope” herein is meant that the amino acids making up the epitope are not in a linear string in the primary structure (e.g. amino acid sequence); rather, the epitope is made up of amino acids from different, non-continuous parts of the sequence, brought together Into spatial proximity by the folding of the native protein, sufficient to form an epitope (e.g. binding site) in the folded form of the protein (e.g. tertiary structure).

The discontinuous epitopes are on a target protein or on protein-protein interacting regions. By “protein” herein is meant at least two covalently attached amino acids, which includes proteins, polypeptides, oligopeptides and peptides. The protein may be made up of naturally occurnng amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. The side chains may be In either the (R) or the (S) configuration. In the preferred embodiment, the amino acids are in the (S) or L-configuration. If non-naturally occurring side chains are used, non-amino acid substituents may be used, for example to prevent or retard in vivo degradation.

“Target protein” in this context means any protein for which a full or partial structure or discontinuous epitope map is desired. Preferred embodiments are those which have associated antibodies or against which antibodies or aptamers can be generated, using methods well known in the art. As will be appreciated by those in the art, there are a wide variety of suitable target proteins which find use in the present Invention including, but not limited to, cell surface receptors, members of signaling systems, metabolic regulation systems, and enzymes (including but not limited to, hydrolases such as proteases, carbohydrases, lipases; isomerases such as racemases, epimerases, tautomerases, or mutases; transferases, kinases and phophatases). Preferred enzymes include those that carry out group transfers, such as acyl group transfers, including endo- and exopeptidases (serine, cysteine, metallo and acid proteases); amino group and glutamyl transfers, including glutaminases, γ glutamyl transpeptidases, amidotransferases, etc.; phosphoryl group transfers, including phosphotases, phosphodiesterases, kinases, and phosphorylases; nucleotidyl and pyrophosphotyl transfers, including carboxylate, pyrophosphoryl transfers, etc.; glycosyl group transfers; enzymes that do enzymatic oxidation and reduction, such as dehydrogenases, monooxygenases, oxidases, hydroxylases, reductases, etc.; enzymes that catalyze eliminations, isomerizations and rearrangements, such as elimination/addition of water using aconitase, fumarase, enolase, crotonase, carbon-nitrogen lyases, etc.; and enzymes that make or break carbon-carbon bonds, i.e. carbanion reactions; suitable enzymes are listed in the Swiss-Prot enzyme database; signaling proteins, cell surface proteins, intracellular proteins, etc. In addition, in some cases the target protein can either be a fragment of a full length protein or a fusion protein, comprising additional sequences, or can be protein-protein interaction regions.

The methods comprise providing a solid support with an attached antibody to the target protein. By “substrate” or “solid support” or other grammatical equivalents herein is meant any material that can be modified to be appropriate for the attachment or association of the antibodies of the invention. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, and a variety of other polymers. The support may take on a variety of geometries, including the use of beads (e.g. affininty chromatography columns), magnetic beads, microtiter plates, etc.

The term “antibody” includes antibody fragments, as are known in the art, Including Fab Fab₂, single chain antibodies (scFv or Fv for example), chimeric antibodies, etc., either produced by the modification of whole antibodies or those synthesized de novo using recombinant DNA technologies. The term “antibody” further comprises polyclonal antibodies and monoclonal antibodies, which can be agonist or antagonist antibodies.

The antibodies of the invention preferably specifically bind to the target proteins. By “specifically bind” herein is meant that the antibodies bind to the protein with a binding constant in the range of at least 10⁻⁴-10⁻⁸ M⁻¹, with a preferred range being 10⁻⁷-10⁻⁹ M⁻¹. Nucleic acid aptamers that bind to target proteins with high affinity can also be used instead of antibodies as described below.

The antibodies may be polyclonal or monoclonal. In addition, it may be desirable to utilize a mixture of antibodies which bind to different discontinuous epitopes, either in a single affinity column, or more preferably in a set of experiments, in order to elucidate more than one of the localized tertiary structures of the target protein. That is, in some cases, it may be preferably to map the active site of the target protein, including enzymatic activity, binding activity, activation activity, etc., and thus chose antibodies that reduce or eliminate the biological function of the target protein.

Monoclonal antibodies are directed against a single antigenic site or a single determinant on an antigen. Thus monoclonal antibodies, in contrast to polyclonal antibodies, which are directed against multiple different epitopes, are very specific. Monoclonal antibodies are usually obtained from the supernatant of hybridoma culture (see Kohler and Milstein, Nature 256:495-7 (1975); Harlow and Lane, Antibodies: A Laboratory Manual (New York: Cold Spring Harbor Laboratory Press, 1988).

In a preferred embodiment, the antibodies to the target proteins are human or are humanized by techniques which are well known in the art.

In a preferred embodiment, aptamers are used instead of antibodies. Nucleic acid “aptamers” can be developed for binding to virtually any target analyte, as is generally described in U.S. Pat. Nos. 5,270,163, 5,475,096, 5,567,588, 5,595,877, 5,637,459, 5,683,867, 5,705,337, and related patents, hereby incorporated by reference. The aptamers comprise nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawal et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids, Koshkin et al., J. Am. Chem. Soc. 120:13252-3 (1998); positive backbones (Denpcy et al., Proc. NaU. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifations in Antisense Research”, Ed. Y. S. Sanghui and P. Dan Cook; Mesmaeker et al., Bloorganic.& Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to increase the stability and half-life of such molecules in physiological environments.

As will be appreciated by those in the art, the antibody or aptamer can be attached to the solid support in a number of ways, including covalent and non-covalent methods, using techniques well known in the art. Preferably, the technique utilized does not mask or sterically hinder the binding region of most of the antibodies used in the experiments.

The solid support is contacted with a library of random peptides under conditions that allow for a set of probe peptides bind to the antibody to the target protein. By “libraries” is meant a plurality. In a preferred embodiment, the libraries provided herein comprise between about 10³ and about 10⁹ independent clones, with from about 10⁵ to about 10⁸ being preferred, and about 10⁵ to about 10⁵ being especially preferred.

By “random peptide” herein is meant peptides that have random sequences. The peptides can be either fully randomized or they are biased in their randomization, e.g. in nucleotide/residue frequency generally or per position. By “randomized” or grammatical equivalents herein is meant that each nucleic acid and peptide consists of essentially random nucleotides and amino acids, respectively. As is more fully described below, in one embodiment, the candidate nucleic acids which give rise to the candidate expression products are chemically synthesized, and thus may incorporate any nucleotide at any position. Thus, when the candidate nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized candidate nucleic acids.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is an unacceptable high likelihood that 46.7% of the peptides will prematurely terminate. For free peptide structures this is perhaps not a problem. But for larger structures, such as those envisioned here, such termination will lead to sterile peptide expression. To alleviate this, random residues are encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the location of cysteines, for cross-linking constraints on peptide conformations, prolines for SH-3 domains, serines, threonines, tyrosines or histidines for phosphorylation sites, etc.

Another type of library, generally referred to herein as “randomized”, are cDNA libraries that have been digested in such a way as to generally be out of frame. In some circumstances, in-frame cDNA digests can be used, which for the purposes of this invention will fall into the definition of “random” as well.

As used herein, the term “cDNA” means DNA that corresponds to or is complementary to at least a portion of messenger RNA (mRNA) sequence and is generally synthesized from an mRNA preparation using reverse transcriptase or other methods. cDNA as used herein includes full length cDNA, corresponding to or complementary in sequence to full length mRNA sequences, partial cDNA, corresponding to or complementary In sequence to portions of mRNA sequences, and cDNA fragments, also corresponding to or complementary to portions of mRNA sequences. It should be understood that references to a particular “number” of cDNAs or other nucleic acids actually refers to the number of clones, cDNA sequences or species, rather than the number of physical copies of substantially identical sequences present. Moreover, the term is often used to refer to cDNA sequences incorporated into a plasmid or viral vector which can, in turn, be present in a bacterial cell, mammalian packaging cell line, or host cell.

By “cDNA fragment” is meant a portion of a cDNA that is derived by fragmentation of a larger cDNA. cDNA fragments may be derived from partial or full length cDNAs. As will be appreciated, a number of methods may be used to generate cDNA fragments. For example, cDNA may be subjected to shearing forces in solution that can break the covalent bonds of the backbone of the cDNA. In a preferred embodiment, cDNA fragments are generated by digesting cDNA with restriction endonuclease(s). Other methods are well known in the art.

“Partial cDNA” refers to cDNA that comprises part of the nucleic acid sequence which corresponds to or is complementary to the open reading frame (ORF) of the corresponding mRNA.

“Full length cDNA” refers to cDNA that comprises the complete sequence which is complementary to or corresponds to the ORF of the corresponding mRNA. In some instances, which are clear, full length cDNA refers to cDNA that comprises sequence complementary to or corresponding to the 5′ untranslated region (UTR) of the corresponding mRNA, in addition to sequence which is complementary to or corresponds to the complete ORF.

The initial mRNA used to generate the libraries may be present in a variety of different samples, where the sample will typically be derived from a physiological source. The physiological source may be derived from a variety of eukaryotic and prokaryotic sources. In addition, viral RNA may be used to serve as template for cDNA synthesis. Physiological sources of interest include sources derived from single celled organisms such as yeast and multicellular organisms, including plants and animals, particularly mammals, preferably humans, primates and rodents, where the physiological sources from multicellular organisms may be derived from particular organs or tissues of the multicellular organism, or from isolated cells derived therefrom. In obtaining the sample of RNAs from the physiological source from which it is derived, the physiological source may be subjected to a number of different processing steps, where such processing steps might include tissue homogenization, cell isolation and cytoplasmic extraction, nucleic acid extraction and the like, where such processing steps are known to those of skill in the art. Eukaryotic and prokaryotic sources include, but are not limited to, bacteria, plant, fungi, insect and mammalian sources, which include, but are not limited to algae, Arabidopsis thaliana, Aspergillus, Axolotl, baboon, bovine, barley, canine, carp, chicken, corn, Drosophila melanogaster, feline, firefly, frog, Fugu fish, hamster, human, lobster, monkey, mouse, nematode, opposum, pea, porcine, rabbit, rat, rice, sea urchin, sheep, soybean, spinach, tobacco, tomato, wheat, Xenopus laevis, yeast, and zebrafish. Preferred sources of RNA for use in the present invention are human, rodent, and primate. Tissue and cell sources for RNA include, but are not limited to, adipose, adrenal, adult brain, adult liver, adult ovary, amygdala, aorta, B-cell, T-cell, mast cell, bladder, blood, bone marrow, brain tumor, breast, breast tumor, capillary endothelial cells, carcinoma, cerebellum, cervix, chondrocyte, colon, colon tumor, colorectal adenocarcinoma, embryo, embryonic brain, embryonic adrenal, embryonic eye, embryonic gut, embryonic liver, embryonic lung, embryonic muscle, embryonic spleen, endothelial, epidermis, epithelial cell, erythroleukemia, esophageal tumor, esophagus, eye, fetus, fetal brain, fetal adrenal, fetal eye, fetal gut, fetal liver, fetal lung, fetal muscle, fetal spleen, fibroblast, fibrosarcoma, glioblastoma, glioma, heart, adult heart, HeLa, hepatocarcinoma, hepatoma, hippocampus, hypothalamus, intestine, small intestine, keratinocyte, kidney, kidney tumor, liver, liver tumor, lung, lung tumor, lymph node, lymphocyte, lymphoblast, lymphoma, macrophage, microglia, mammary gland, mucus-producing gland, muscle, myoblast, monocyte, nasal mucosa, neuronal, NIH 3T3, stomach, thyroid, uterus, oocyte, pancreas, ovarian tumor, pituitary, prostate, rectal tumor, rectum, retina, salivary gland, spinal cord, spleen, submucosa, stem cell, and tonsil. Viral nucleic acids may also be used.

Once isolated, mRNAs are then used as template for the synthesis of double stranded cDNA (dscDNA) using the enzyme reverse transcriptase. Synthesis of cDNA may be done in vitro or in vivo, as is known (for example, see U.S. Pat. No. 5,891,637, issued 6 Apr. 1999 to Ruppert et. al, incorporated herein be reference).

Whether random, biased, synthetic, or derived from natural sources, in general, the library should provide a sufficiently structurally diverse population of randomized expression products to effect a probabilistically sufficient range of peptide sequences to match to the target protein antibody or aptamer epitope. Accordingly, an interaction library must be large enough so that at least one of its members, and preferably a set, will have a structure that gives it affinity for the antibody to the target protein. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, such as proposed here, has the potential to code for 20⁷ (10⁹) to 20²⁰. Thus, with libraries of 10⁷ to 10⁸ per ml of solution the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different expression products are simultaneously analyzed in the subject methods. Preferred methods maximize library size and diversity.

In a preferred embodiment, the random peptides are linked to a fusion partner. By “fusion partner” or “functional group” herein is meant a sequence that is associated with the peptide that confers upon all members of the library in that class a common function or ability. Fusion partners can be heterologous (i.e. not native to the host cell), or synthetic (not native to any cell). In a preferred embodiment, the fusion partner is a phage display scaffold. Alternatively, suitable fusion partners include, but are not limited to: a) presentation structures, as defined below, which provide the peptides in a conformationally restricted or stable form; b) rescue sequences as defined below, which allow the purification or isolation of either the peptide or the nucleic acids encoding them; c) stability sequences, which confer stability or protection from degradation to the peptide or the nucleic acid encoding it, for example resistance to proteolytic degradation; d) dimerization sequences, to allow for peptide dimerization; e) cyclization sequences, such as cysteine residues at the termini; or f) any combination of a), b), c), d), and e), as well as linker sequences as needed.

In a preferred embodiment, the fusion partner is a presentation structure. By “presentation structure” or grammatical equivalents herein is meant a sequence, which, when fused to the peptide libraries of the invention, causes the peptides to assume a conformationally restricted form. Proteins interact with each other largely through conformationally constrained domains. Therefore the presentation of peptides in conformationally constrained structures will likely lead to higher affinity interactions of the peptide with the target antibody. This fact has been recognized in the combinatorial library generation systems using biologically generated short peptides in bacterial phage systems. A number of workers have constructed small domain molecules in which one might present randomized peptide structures. In other cases the lack of conformational constraint of linear peptide libraries is preferred so that the library members can better conform to the antibody or aptamer binding pockets.

Suitable presentation structures include, but are not limited to, phage display systems, peptide cyclization systems including the use of disulfides, minibody structures, loops on beta-sheet turns and coiled-coil stem structures in which residues not critical to structure are randomized, zinc-finger domains, cysteine-linked (disulfide) structures, transglutaminase linked structures, B-loop structures, helical barrels or bundles, leucine zipper motifs, etc.

In a preferred embodiment, the fusion partner is a rescue sequence. A rescue sequence is a sequence which may be used to purify or isolate either the peptide or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the fusion partner is a stability sequence to confer stability to the peptide or the nucleic acid encoding it. Thus, for example, peptides may be stabilized by the incorporation of glycines after the initiation methionine (MG or MGG0), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines Impart both flexibility and prevent structure initiating events In the di-proline to be propagated into the candidate peptide structure. Thus, preferred stability sequences are as follows: MG(X)ₙGGPP, where X is any amino acid and n is an integer of at least four. In one embodiment, the fusion partner is a dimerization sequence. A dimerization sequence allows the non-covalent association of one random peptide to another random peptide, with sufficient affinity to remain associated under normal physiological conditions. This effectively allows small libraries of random peptides (for example, 10⁴) to become large libraries if two peptides per cell are generated which then dimerize, to form an effective library of 10⁸ (10⁴×10⁴). It also allows the formation of longer random peptides, if needed, or more structurally complex random peptide molecules. The dimers may be homo- or heterodimers.

Dimerization sequences may be a single sequence that self-aggregates, or two sequences. That is, nucleic acids encoding both a first random peptide with dimerization sequence 1, and a second random peptide with dimerization sequence 2, such that upon introduction into a cell and expression of the nucleic acid, dimerization sequence 1 associates with dimerization sequence 2 to form a new random peptide structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

The fusion partners may be placed anywhere (i.e. N-terminal, C-terminal, internal) in the structure as the biology and activity permits.

In a preferred embodiment, the fusion partner includes a linker or tethering sequence. Linker sequences between various components may be useful to allow the peptides to interact with potential target antibodies unhindered. For example, useful peptide linkers include glycine-serine polymers (including, for example, (GS)ₙ, (GSGGS)ₙ and (GGGS)ₙ, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In a preferred embodiment, combinations of fusion partners are used.

Once a library of peptides has been generated, the library is used in the antibody imprint method. The core idea of the antibody imprint method is that a probe (e.g. random peptide) that binds to the active region of a particular antibody (e.g. the antigen binding portion) is expected to be highly similar to the binding site of a protein that also binds to the same antibody. Essentially, a “positive” (the target protein) is used to make a “negative” (e.g. the antibody) which is used to recreate a new “positive” (the epitope). Alternatively, the negative can be made of single-stranded nucleic acid aptamers. Thus the invention deals with the problem of aligning the probe amino acid sequence, s, to one or more regions of the target protein amino acid, t. In this context, “alignment” is not used as for aligning homologous nucleic acid or amino acid systems. Rather, “alignment” in this context is the mapping of the physical surface of the probe to the physical surface of the target as a binding site of the two.

Typically, s is about 8-20 amino acids long and t is several hundred. Unlike traditional string alignment problems, localized sequence inversions and rearrangements are allowed. This captures the possibility that several loops of the linear protein sequence may be pinched together (possibly with sequence inversions) to form the binding site. Additionally, it is possible for local rearrangements of amino acids to occur, reflecting the fact that the binding site of an antibody is a surface, not just a linear sequence. As such, the problem is outside the scope of classical string alignment algorithms such as Smith-Waterman [Smith and Waterman, 1981]. The present invention is directed to an approach based on a general combinatorial alignment problem, although alternative strategies such as hidden Markov models and stochastic free grammars have been employed for related problems and could be utilized within the present invention.

In one embodiment of the invention, the antibody to a target protein is contacted with a library of random peptides under conditions where a set of the probe peptides will bind to the antibody. As described above, the probe peptides are eluted from the antibody and the amino acid sequences of the members of the set of probe peptides are determined. The probe sequences are then computationally aligned with the target protein amino acid sequence and the resulting alignment is used to construct a surface epitope on the target protein.

In a preferred embodiment, a consensus epitope sequence is determined from the amino acid sequences of the members of the set of probe peptides. The consensus epitope sequence is then computationally aligned with the target protein and the resulting alignment is used to construct a surface epitope on the target protein.

In a further preferred embodiment, a surface epitope of a target protein is constructed using a graph-based approach. This approach eliminates the step of finding a consensus epitope sequence. Typically 25-100 peptide probes are sequenced that show strong affinity to the antibody in question. These sequences are often rather similar, but typically not identical. The center of the antibody combining site is expected to contribute to the highest probe affinity [Conte et al., 1999], whereas more peripheral binding site residues tend to make a lower contribution to the affinity, and thus may show alternative binding modes. Rather than going through the step of finding a consensus sequence, an alignment algorithm or program, such as FINDMAP, can be run on each of the probe sequences individually to generate a family of top-scoring alignment sets, one set for each probe. These alignments are similar, but often indicate the proximity of additional residues on the protein surface. A graph-based approach can be used to merge and visualize the collective surface proximity information provided an entire set of alignments. In this approach each residue of the target protein constitutes a vertex in a weighted surface-neighbor graph. Edge weights in this graph indicate how strongly the epitope mapping data supports the conclusion that the residues at each endpoint are neighbors on the surface of the protein.

The specific procedure employed for calculating edge weights is as follows: for each probe, the set of top scoring alignments is computed. The method supposes there are n such alignments and that a particular pair of residues are neighbors in k of these alignments. Then k/n is added to the weight of the edge between the two residues in question. After this procedure is repeated for each probe, edges that have comparatively high weights are most likely to link residues that are true surface neighbors. In practice, errors occur both in the experimental methods used to identify the probe sequences as well as cases where the top-scoring alignments are not biologically accurate. Thus it appears useful to use a weight cutoff; edges are only kept if their weight is greater than a prespecified cutoff. If the cutoff is too low, it is likely that false surface neighbor relations will be included in the graph; too high and true neighbors will be lost. Another procedure that is useful for pruning out non-epitope residues from the surface neighbor graph is to retain only vertices that are incident to at least one high-weighted edge. The surface neighbor graph can be used to make a map of the surface of the protein.

As discussed above, the probe peptides are computationally aligned with the target protein to construct a surface epitope. By “computationally aligned” is meant the use of an algorithm or program to align the probe peptide sequence to the target protein to construct surface epitopes.

In general, any permutation of the probe sequence to align to the underlying protein sequence is allowed. Furthermore, gaps are permitted in both probe and target sequences. Large gaps can occur when aligning the probe to the target sequence when the epitope is discontinuous. In addition, unaligned probe residues are also aligned, reflecting the possibility of a non-specific residue insertions in the probe. To be a valid alignment, each probe position and target position can be used at most once per mapping. Formally, an alignment A consists of a sorted set P_{A}={i₁<i₂< . . . <iₖ}, and another T_{A}={j₁, j₂, . . . , jₖ}, with the interpretation that the iₚ-th probe residue, s(iₚ), is aligned to the jₚ-th target residue t(jₚ), for 1≦p≦k.

A two-part scoring system is used to evaluate the quality of alignments. The scoring system is composed of a substitution score and a epitope gap cost,
score(A)=S(A)−G(A).

The S(A) component is calculated with a substitution matrix M, similar in principle to a Dayhoff matrix, used in other protein alignment contexts. The substitution matrix is also used to score unaligned probe residues; if the probe residue in position I is not aligned to any target position then it is charged a penalty according to the character occurring in position i of the probe sequence. This cost can be found in the substitution matrix, in the entry M(c, −). We have

$$S ( A ) = {\sum }_{p = 1}^{k} M ( s ( {i}_{p} ) , t ( {j}_{p} ) ) + {\sum }_{probe positions i ∉ {P}_{A}} M ( s ( i ) , - )$$

The epitope gap cost G(A) is calculated by examining the number of amino acid residues skipped along the target protein sequence between successive aligned probe positions:

$$G ( A ) = {\sum }_{p = 1}^{k - 1} d [ \vert {j}_{p + 1} - {j}_{p} \vert ]$$

where d(x) is the cost of skipping x amino acids along the target between successive mapped probe positions. For circular probes we also include the term d[|jₖ−jₗ|] in the above sum. The computational problem is thus to finding an alignment A that maximizes score (A).

As will be appreciated by those in the art, there are a number of suitable computational methods that can be applied to find an alignment. A preferred embodiment utilizes a branch-and-bound algorithm or program.

A branch-and-bound algorithm or program can be used to solve this alignment problem in practice. The algorithm constructs a search tree to find the optimal alignment(s). Often, a user may also be interested in near-optimal solutions so the algorithm is designed to find the top r solutions where r is user-specified. Each node in the search tree represents a partial alignment of the probe to the protein sequence. At the root, all probe positions are unaligned. Nodes at level i>0 in the tree fix the alignment of the i-th probe position (either to an available target position or to a “−”, indicating an unmatched probe position). A leaf is reached when all probe positions have been considered and each leaf represents a particular alignment. Whenever a new node n is created, an upper bound on the highest possible alignment score achievable in the subtree rooted at n is computed. If this bound is less than the r-th best solution found so far, we can immediately prune the node from the search. Nodes that are on the boundary of current search tree are said to be on the frontier. For each frontier node n, an expected score is calculated by dividing n's current score by its depth in the tree. A heap data structure is used to extract a node with maximal expected score from the frontier. This node is then expanded; descendant child nodes are created for each possible alignment of the next probe position. When a leaf is reached, the score of the associated alignment is calculated. This score is compared to the current r-th best solution and if greater replaces it. When such a replacement occurs, the frontier is scanned to cull out any other nodes that can now be eliminated. This algorithm has been implemented as a C++ program called FINDMAP.

In one embodiment, the detailed conformation of the mapped epitope is determined by means known to those of skill in the art. For example, the conformation of the probe peptides bound to the antibody or aptamer are determined using nuclear magnetic resonance (NMR) methods. The probe peptides with the antibody or aptamer can be co-crystallized and the structure resolved using X-ray crystallography or other such means.

It is to be understood that the methods may be implemented in hardware, software, or any combination thereof. Furthermore, the invention may advantageously implement the methods and procedures described herein on a general purpose or special purpose computing device, such as a device having a processor for executing computer program code instructions and a memory coupled to the processor for storing data and/or commands. It will be appreciated that the computing device may be a single computer or a plurality of networked computers and that the several procdures associated with implementing the methods and procedures described herein may be implemented on one or a plurality of computing devices. In some embodiments the inventive procedures and methods are implemented on standard server-client network infrastructures with the inventive features added on top of such infrastructure or compatible therewith.

The system used can include a computer workstation comprising a microprocessor programmed to manipulate a device selected from the group consisting of a thermocycler, a multichannel pipettor, a sample handler, a plate handler, a gel loading system, an automated transformation system, a gene sequencer, a colony picker, a bead picker, a cell sorter, an incubator, a light microscope, a fluorescence microscope, a spectrofluorimeter, a spectrophotometer, a luminometer, a CCD camera and combinations thereof.

It is to be understood that all the steps in the antibody imprinting process are adaptable to high throughput enhancement. High throughput enhancements can be applied to, for example, epitope selection, epitope sequencing, and epitope mapping. In some cases suitable antibodies are already available, but in many cases suitable antibodies have not been prepared or do not provide sufficient coverage of the protein surface or the conformational states of interest. In the absence of available antibodies, the rate-limiting step in the current process is the isolation and characterization of new antibodies. Technology to express random antibody libraries on phage [Pini et al., 1998, Hoogenboom et al., 1998] has been developed and shows promise for much more rapid identification and preparation of specific antibodies. Affinity maturation steps applied to antibodies selected from random libraries to obtain subpicomolar antibody affinities [Pini et al., 1998] may also be adaptable to high throughput approaches. Random antibody libraries appear to be uniquely useful for rapid selection against transient protein conformations, which are expected to reveal important information on protein mechanisms.

In addition, as will also be appreciated by those in the art, the inventive procedures and methods may be part of tigh throughput screening (HTS) system utilizing any number of components. Fully robotic or microfluidic systems include automated liquid-, particle-, cell- and organism-handling including high throughput pipetting to perform all steps of gene targeting and recombination applications. This includes liquid, particle, cell, and organism manipulations such as aspiration, dispensing, mixing, diluting, washing, accurate volumetric ransfers; retrieving, and discarding of pipes tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

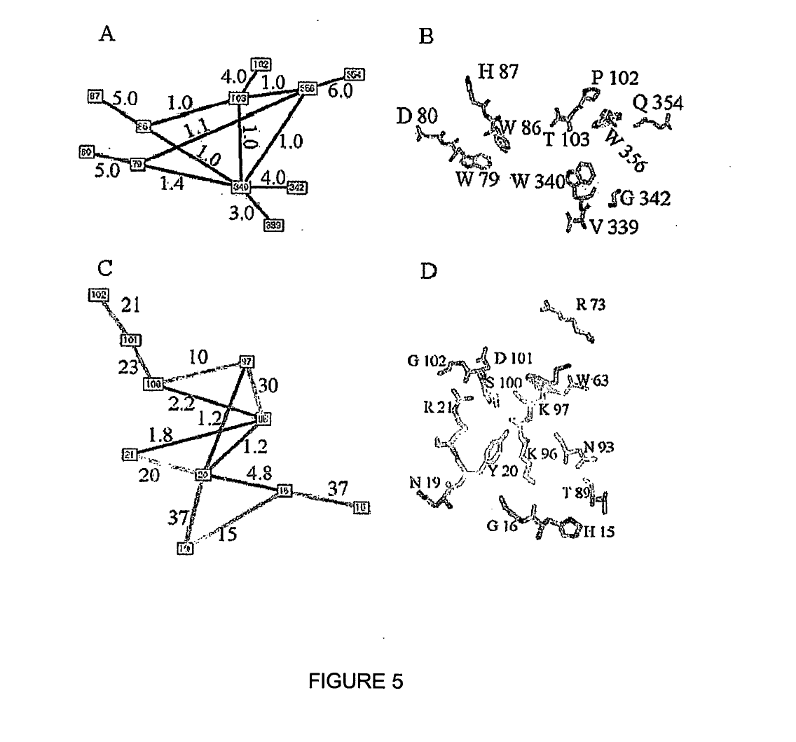

The present invention may also be adapted to using mixtures of selected random antibody library members, avoiding the growth and screening of individual phage clones. Given a set of a probe-target alignments, it is possible that they be clustered into groups corresponding to unique epitopes of the target protein. Probe-target alignments can be evaluated for each probe found that binds at least one of these antibodies. These probe-target alignments are then clustered into putative epitope groups and a confidence value for each epitope is predicted. It may be possible to simultaneously collate all of probe-target alignments to produce a surface-neighbor graph that contains (possibly disconnected) clusters for each epitope present in a similar way as the example shown in FIG. 5, but perhaps with a much larger surface coverage.

### EXAMPLES

### Example 1

### Probe-Target Sequence Alignment Problem is NP-Complete

In this section we show that the probe-target sequence alignment problem is NP-complete [Garey and Johnson, 1979]. In computational complexity theory, NP (“non-deterministic polynomial-time”) is the set of decision problems solvable in polynomial time on a non-deterministic Turing machine. Or, equivalently, the set of decision problems that can be reformulated as a binary function A(x, y) over strings such that for a certain constant number c it holds that a string x is an element of the original decision problem if there is a string y with length smaller than |x|c such that A(x, y), the function A is decidable by a Turing machine in polynomial time. An y value for a certain x such that A(x, y) holds is usually referred to as a certificate for x since it shows the membership of x to the original decision problem. Informally, a problem is NP-complete if answers can be verified quickly, and a quick algorithm to solve this problem can be used to solve all other NP problems quickly.

A decision version of the problem is first defined:

The ALIGN decision problem.

Input: A probe string s, a target string t (over a common alphabet), a substitution score matrix M, a distance penalty function d, an objective score Q.

Output: A decision on whether there exists an alignment with score at least Q.

Lemma 1 ALIGN is NP-complete.

Proof. First note that ALIGN belongs to NP because the score of a given alignment can be checked in polynomial time. It can be shown that ALIGN is complete for NP via a polynomial time reduction from 3SAT. Consider an instance of 3Sat I_{3S} consisting of a collection of clauses C={c₁, c₂ . . . , cₘ} on a finite set of variables U={x₁, . . . , xₖ}. A polynomial time reduction can be described to an instance I_{A}=(s,t,M,d,Q) of ALIGN such that a truth assignment exists for U that satisfies C if and only if an alignment between s and t with score at most Q can be found. I_{A} is constructed as follows.
A=U∪{ [symbol] x₁, . . . [symbol] xₖ}∪{y₁, . . . , yₖ}
∪{‘c₁’, . . . , ‘cₘ’}∪{‘#’, ‘*’, ‘@’}.

All entries of Mare set to −∞ except the following: M(α,cᵢ)=0 if α is a literal in clause cᵢ,M(χᵢ,yᵢ)=M( [symbol] χᵢ,yᵢ)=0 for all 1≦i≦n, and M(., ‘*’)=0 (

For each literal α, let [α]be the multiplicity of α among all clauses in C. The probe string used is
s=@B₁B₂ . . . Bₖ

where

$$a . {B}_{i} = {\underbrace{{x}_{i} ⋯ {x}_{i}}}_{[ {x}_{i} ] + 1 copies } @ {\underbrace{⫬ {x}_{i} ⋯ ⫬ {x}_{i}}}_{[ ⫬ {x}_{i} ] + 1 copies} @ .$$

Let n=|s|−(m+k). The target string used is

$$t = \underbrace{** … *}_{{n}^{copies}} \underbrace{\#\# … \#}_{{n}^{copies}} {c}_{1} {c}_{2} ⋯ {c}_{m} {y}_{1} {y}_{2} ⋯ {y}_{k} .$$

The distance penalty function used is

$$d ( l ) = \{ \begin{matrix}0 & if l < n \\ 1 & otherwise .\end{matrix}$$

Observe that m+k<n, so only jumps across the central gap of #'s, referred to as the bridge, will contribute to the gap cost. The leading @ of s forces any finite-score alignment to begin on the left side of the bridge. Note that every non-# letter in the target must be matched in order to completely align the probe (all probe positions must be matched as M(·, −)=−∞). In order to match all of the yᵢ, at least one literal from each Bᵢ must be used. Thus each Bᵢ contributes at least one return jump across the bridge. If a literal is matched against a clause symbol cᵢ, then any truth assignment that makes this literal true will satisfy cᵢ. We choose Q=−2k to insist that each Bᵢ contributes exactly one return jump across the bridge. Because the positive and negative literals in each block Bᵢ are separated by an @, only literals of a single polarity can be matched to symbols to the right of the bridge. This ensures a consistent truth assignment. Thus, any alignment with score exactly −2k will produce a satisfying assignment for I_{3S} and vice versa.

### Example 2

### Implementation of the Branch and Bound Alignment Algorithm—Validation using Actin Model

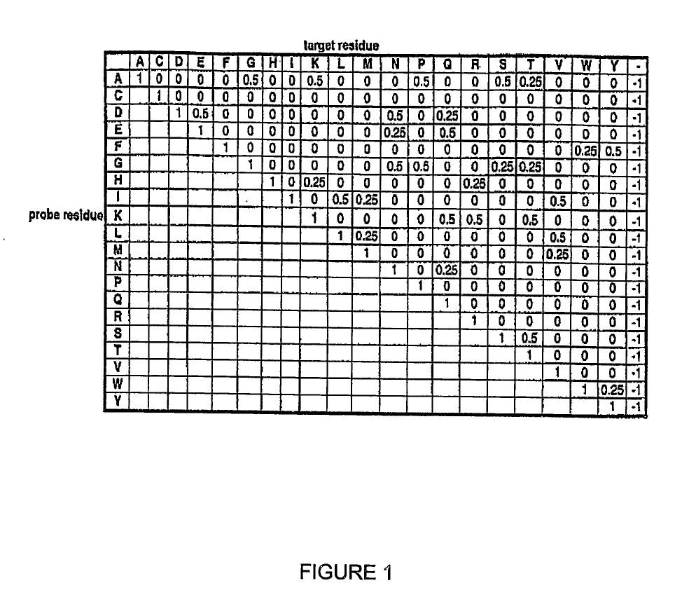

This Example discusses experimental results using FINDMAP and the implementation of the branch-and-bound alignment algorithm described above to a protein of known structure: actin. FINDMAP requires an amino acid substitution probability matrix to score sequence alignments. The matrix shown in FIG. 1 was chosen for initial mapping, since a very similar substitution matrix was developed by Bordo and Argos [Bordo and Argos, 1991, incorporated by reference herein] for scoring substitutions of protein residues exposed to the aqueous surface. Antibody binding sites on target proteins must be exposed to the aqueous surface for antibody accessibility and so an aqueous-exposed substitution seems appropriate. Experimental substitution matrixes can be optimized for antibody imprinting using training cases with known antibody-protein or aptamer-protein complex structures and the known antibody or aptamer epitopes.

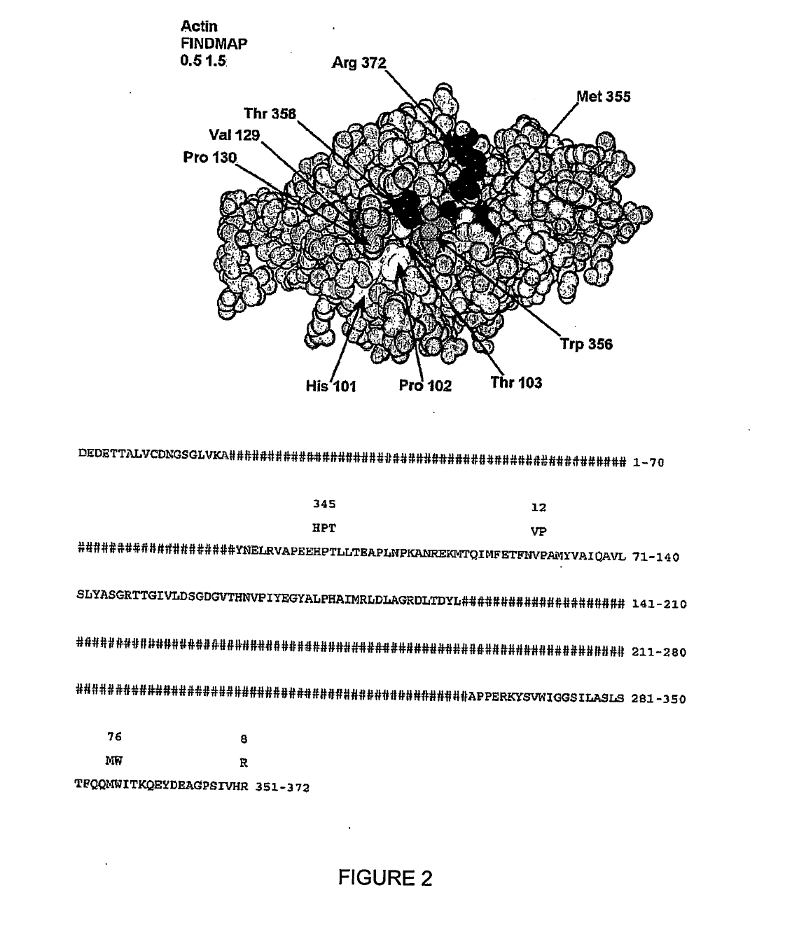

Recently, Jesaitis and co-workers carried out antibody Imprinting using a polyclonal antibody against the ubiquitous cytoskeletal protein, actin [Jesaitis et al., 1999]. They reported the manual mapping of consensus peptides derived from phage display library selection, to complex epitopes on the surface of actin. The phage-display-discovered peptides could be mapped onto the actin surface to mimic a discontinuous epitope that was consistent with the known 3-D x-ray structure of actin [Kabsch et al., 1990]. FIG. 2 shows the mapping of one of the consensus sequences, VPHPTWMR, onto the surface of actin and the almost identical FINDMAP mapping. It should be emphasized that this manual mapping utilized knowledge of the actin x-ray structure and did not use residues marked with # that are not exposed on the aqueous surface in the x-ray structure.

The FINDMAP alignment used only the protein primary sequence. The single difference from the manual mapping is FINDMAP's selection of the more buried but plausible Thr 103 instead of the more exposed Thr 358 for the T in VPHPTWMR. This result is an initial validation of the antibody Imprinting technique and FINDMAP on a known protein structure. Additional validation studies are described in a later section. It is possible that including an estimate for the probability of surface exposure in the overall alignment scoring function could be useful [Jameson and Wolf, 1988] and this is being explored further.

The actin test case was used to optimize the gap cost parameters for gaps in the alignment of the target protein sequence to the probes. A simple linear penalty function up to a maximum gap penalty that does not further penalize long gaps (long gaps are expected to be frequent in discontinuous epitopes) was chosen:
d(n)=min(a·n, b)

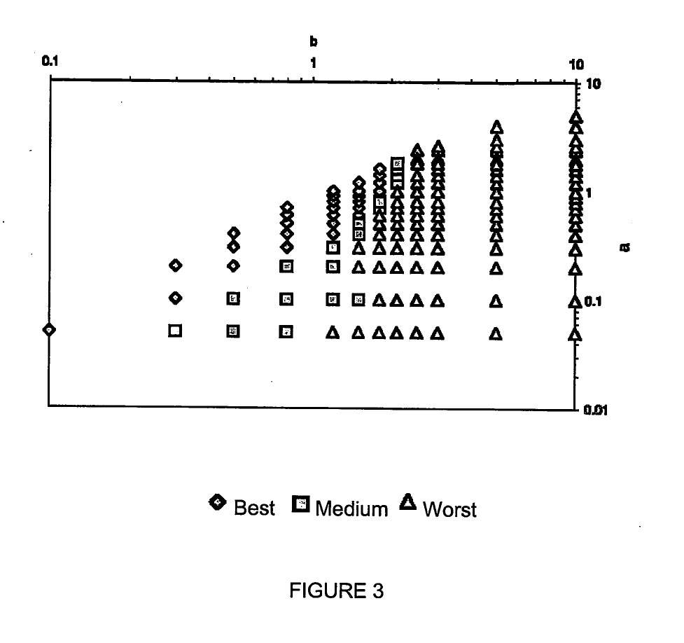

To search for suitable values of a and b, we ran FINDMAP on the actin example, where the 3-D structure is known, using the probe sequence VPHPTWMR. 140 different combinations of a, b pairs were tested, as shown in FIG. 3. The deviation from the proper mapping with parameter values that were non-optimal were systematic. When a was set too small, the highest scoring epitopes found were implausibly discontinuous with identity matches widely spread in the mappings at the expense of any allowable amino acid substitutions.

In contrast, when a was too large, excessively continuous local epitopes were found, that may include large numbers of very non-favorable amino acid substitutions. In FIG. 3, the best parameter choices yielded 18 alignments that had identical optimal scores, of which one agreed exactly with the manual mapping except at one residue position, as described in the caption to FIG. 2. A reason for the proliferation of near optimal solutions In this case is the freedom of the final R in the consensus probe to align to a number of positions in the target). The combination of a=0.5 and b=1.5 was chosen from the best region for the subsequent experiments to be discussed.

### Example 3

### Implementation of the Branch and Bound Alignment Algorithm—Mapping of Rhopdopsin

This Example discusses experimental results using FINDMAP and the implementation of the branch-and-bound alignment algorithm described above the integral membrane protein rhodopsin, the structure of which is not fully known. Rhodopsin Is the photoreceptor for dim light vision in retinal rod cells and is an archetype for the structure and mechanism of a large superfamily of cellular G protein-coupled receptor (GPCR) proteins that re-spond to a wide range of hormones and neurotransmitters [Wess, 1997, Marinissen and Gutkind, 2001]. The xray crystal structure of the dark-adapted, resting structure of rhodopsin was recently published [Palczewski et al., 2000, Teller et al., 2001] but some of the features of the protein on the cytoplasmic surface were poorly ordered in the crystals and not visible in the x-ray structure. A computational model of the missing portions of the cytoplasmic surface was built and energy minimized [Bailey, 2001]. The cytoplasmic surface structure was uncertain in the model, so antibody imprinting was applied to the aqueous surfaces [Bailey et al., 2001, 2003].

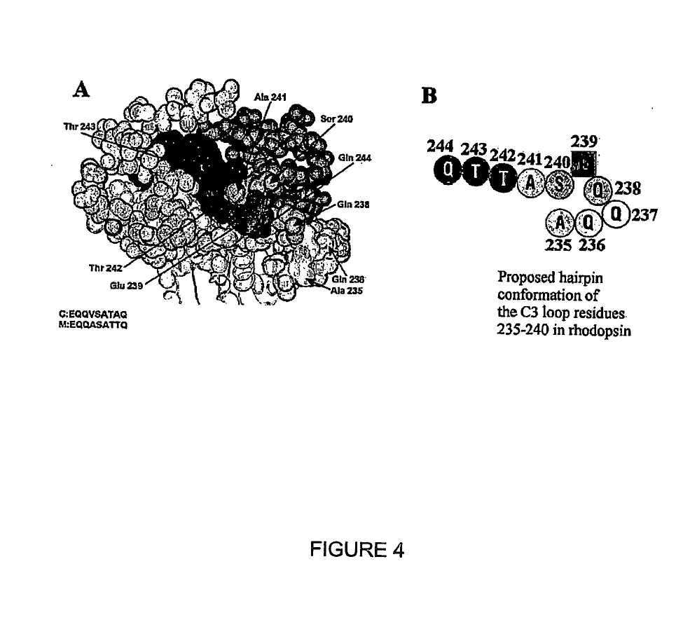

One of the antibodies investigated (B1gN) maps to the extracellular surface of rhodopsin in a compact patch that shows the proximity of two distant segments of sequence, that is in excellent agreement with a well defined region of the x-ray structure [Bailey et al., 2001, 2003] (data not shown). One of the other antibodies studied (4B4) targeted part of rhodopsin where the x-ray structure was not fully resolved, and a model of this region is shown in FIG. 4A. The single most optimal mapping of the 4B4 epitope found with FINDMAP was unusual in that it was continuous with a segment of the rhodopsin sequence. The optimal mapping, however, showed a spatial discontinuity in the proximity of two parts of the epitope as illustrated in FIG. 4. The aligned epitope is shown in FIG. 4B. The Ser 240 residue is predicted by FINDMAP to be located spatially adjacent to the Ala 235 residue but there is a large jump in the structural model, as shown in FIG. 4A. This is evidence that the surface loop folding model shown in FIG. 4A is incorrect and should be adjusted to form a hairpin turn bringing Ala 235 next to Ser 240, as shown in FIG. 4B. This example supports that notion that the antibody imprinting technique is capable of providing new structural information. Experiments are in progress to obtain more detailed conformational information by crystallizing epitope-mimetic peptides with the active site of the antibodies, to provide detailed folding information on regions of the protein surface (Lawrence, Bailey and Dratz, unpublished). The x-ray crystallography appears to be straightforward for co-crystals of peptides with antibody active sites, since molecular replacement with known antibody structures should provide the phases.

Additional antibodies will be required to reveal the complete surface structure of rhodopsin and its light excited conformations. More detailed antibody imprinting studies, seeking to deduce light-stimulated conformational changes in rhodopsin are in progress and some of these have been submitted for publication [Bailey et al., 2003]. Most of the antibody epitopes are found to be discontinuous and thus provide important long-range distance constraints on the structures. If regions of the surfaces studied are flexible it is anticipated that a range of conformations will be deduced by different antibody epitopes consistent with that flexible structure, as would be found if other structural techniques such as NMR or x-ray diffraction could be applied.

It should also be noted that it is possible to include additional information, such as from structure prediction algorithms or experimental information, if available, from intramolecular cross-links identified by mass spectrometry [Young et al., 2000] or from site-specific spin labeling [Hubbell et al., 2000] to add to the information obtained from FINDMAP or to prioritize alternative FINDMAP spatial proximity mappings.

### Example 4

### A Graph-based Approach to Surface Epitope Mapping

An alternative approach to the overall process is to eliminate the step of finding a consensus epitope sequence and to generate a surface neighbor probability graph. modes. Rather than going through the step of finding a consensus sequence, FINDMAP can be run on each of the probe sequences individually to generate a family of top-scoring alignment sets, one set for each probe. These alignments are similar, but often indicate the proximity of additional residues on the protein surface. A graph-based approach was used to merge and visualize the collective surface proximity information provided in the entire set of alignments. In this approach each residue of the target protein constitutes a vertex in a weighted surface-neighbor graph. Edge weights in this graph indicate how strongly the epitope mapping data supports the conclusion that the residues at each endpoint are neighbors on the surface of the protein.

The specific procedure employed for calculating edge weights is as follows: for each probe, compute the set of top scoring FINDMAP alignments. Suppose there are n such alignments and that a particular pair of residues are neighbors in) of these alignments. Then k/n is added to the weight of the edge between the two residues in question. After this procedure is repeated for each probe, edges that have comparatively high weights are most likely to link residues that are true surface neighbors. In practice, errors occur both in the experimental methods used to identify the probe sequences as well as cases where the top-scoring alignments are not biologically accurate. Thus it appears useful to use a weight cutoff; edges are only kept if their weight is greater than a prespecified cutoff. If the cutoff is too low, it is likely that false surface neighbor relations will be included In the graph; too high and true neighbors will be lost. Another procedure that seems useful for pruning out non-epitope residues from the surface neighbor graph is to retain only vertices that are incident to at least one high-weighted edge. This procedure was performed on the surface neighbor graphs shown in FIG. 5, only vertices incident to an edge of weight at least 50% of the maximum weight were kept. Also, a cutoff value of 1 was used to prune low weight edges.

The target sequence is also scanned for multiple occurrences of tripeptide (very rare) or dipeptide sequences in the probe and hits involving these ambiguous sequences are omitted from consideration to minimize false positive hits. It is important to eliminate false positive residue proximity information to provide accurate structure, whereas false negatives are more tolerable. An example of a surface neighbor graph based on actin FINDMAP alignment data of individual probes is shown in FIG. 5A. The somewhat larger protein surface mapped with this approach, compared to FIG. 2, is consistent with the fact that the antibody investigated is polyclonal. Monoclonals that we have primarily used in this work provide surface maps with a smaller area coverage, but it has been found feasible to map mixtures of several monoclonals in parallel in a single experiment (Bailey and Dratz, unpublished).

The surface neighbor graph can be used to make a map of the surface of the protein. The protein surface is two-dimensional, so it seems feasible to consider planar embeddings of the surface neighbor probably graph that place residue vertices in such a way that heavily weighted edges connect neighboring vertices in the embedding. Another criteria is that residues should be packed in a roughly uniform way, in lattices and/or proportional to their molecular volume. FIG. 5 was generated using the program Graphlet, available at www.fmi.uni-passau.de/Graphlet, but alternative methods are possible to perform the graph embeddings. A further important constraint is that residues that are consecutive in the linear protein sequence must also necessarily be neighbors in the embedded surface map. Other related problems that might be useful for protein surface mapping from antibody epitope data include maximum planar subgraph and minimum edge distance graph layout.

3-D structures of the antibody-target protein complex that are known to atomic resolution by x-ray diffraction can be used to more thoroughly validate the accuracy of the surface-mapping method. In these validation cases the correct antibody epitope mappings are known. The first case we investigated is Hen Egg Lysozyme complexed with several different monoclonal antibodies. The antibody contacts on the lysozyme surface have been identified (using the CCP4 Contacts program, http://www.ccp4.ac.uk/main.html). A collection of 50 hypothetical probe sequences were generated by randomly connecting adjacent residues on the lysozyme contact surface on the Hyhel-10 antibody. FINDMAP alignments were found to all the probes generated and the epitope surface neighbor graph was found, using the method described above. In FIG. 5C we show the computed surface neighbor graph and the true epitope surface for monoclonal antibody Hyhel-10 (PDB:1C08). Also shown in FIG. 5D is a diagram of the experimental monoclonal antibody epitope, that is seen to agree favorably with the surface neighbor graph edge weights.

All references are incorporated herein in their entirety.

### REFERENCES

[Bailey, 2001] Bailey, B. (2001). Antibody Imprinting Studies of Rhodopsin, A Model of G Protein-coupled Receptor. PhD thesis, Montana State University.
[Bailey et al., 2003] Bailey, B., Mumey, B., Hargrave, P., Arendt, A., Ernst, O., Hofmann, K., P. Callis, Burritt, J., Jesaitis, A., and Dratz, E. (2003). Structural constraints on the conformation of the cytoplasmic face of dark-adapted and light-excited rhodopsin inferred from anti-rhodopsin antibody imprints, submitted. Protein Science.
[Baker and Sali, 2001] Baker, D. and Sali, A. (2001). Protein structure prediction and structural genomics. Science, 294(5540):93-96.
[Barbas et al., 2001] Barbas, C., Burton, D., Scott, J., and Silverman, G. (2001). Phage Display: a Laboratory Manual. Cold Spring Harbor Laboratory Press.
[Bordo and Argos, 1991] Bordo, D. and Argos, P. (1991). Suggestions for “safe” residue substitutions in sitedirected mutagenesis. J. Mol. Biol., 217:721-729.
[Branden and Tooze, 1999] Branden, C. and Tooze, C. (1999). Introduction to Protein Structure. Garland Publishing.
[Burritt et al., 1996] Burritt, J., Bond, C., Doss, K., and Jesaitis, A. (1996). Filamentous phage display of oligopeptide libraries. Anal. Biochemistry, 238:1-13.
[Burritt et al., 1998] Burritt, J., Busse, S., Gizachew, D., Dratz, E., and Jesaitis, A. (1998). Antibody imprint of a membrane protein surface: Phagocyte flavocytochrome b. J. Biol. Chem., 273:24847-24852.
[Burritt et al., 2001] Burritt, J., DeLeo, F., McDonald, C., Prigge, J., Dinauer, M., Nakamura, M., Nauseef, W., and Jesaitis, A. (2001). Phage display epitope mapping of human neutrophil flavocytochrome b558: Identification of two juxtaposed extracellular domains. J. Biol. Chem., 276:2053-2061.
[Cavanagh et al., 1996] Cavanagh, J., III, A. P., Fairbrother, W., and Skelton, N. (1996). Protein Nmr Spectroscopy: Principles and Practice. Academic Press.
[Clayerie, 2001] Clayerie, J. (2001). What if there are only 30,000 human genes? Science, 291:1255-1257.
[Clore et al., 1993] Clore, G., Robien, M., and Gronenborn, A. (1993). Exploring the limits of precision and accuracy of protein structures determined by nuclear magnetic resonance spectroscopy. J. Mol. Biol., 231:82-102.
[Conte et al., 1999] Conte, L., Chothia, C., and Janin, J. (1999). The atomic structure of protein-protein recognition sites. J. Mol. Biol., 285:2177-2198.
[Dandekar and Argos, 1997] Dandekar, T. and Argos, P. (1997). Applying experimental data to protein fold prediction with the genetic algorithm. Protein Engineering, 10:877-893.
[Demangel et al., 2000] Demangel, C., Maroun, R., Rouyre, S., Bon, C., Mazie, J., and Choumet, V. (2000). Combining phage display and molecular modeling to map the epitope of a neutralizing antitoxin antibody. Eur. J. Biochem., 267:2345-2353.
[Edwards et al., 2000] Edwards, A., Arrowsmith, C., Christendat, D., Dharamsi, A., Friesen, J., Greenblatt, J., and Vedadi, M. (2000). Protein production: feeding the crystallographers and nmr spectroscopists. Nat Struct. Biol., pages 970-972.
[Eisenstein et al., 2000] Eisenstein, E., Gilliland, G., Herzberg, O., Moult, J., Orban, J., Poljak, R., Banerjei, L., Richardson, D., and Howard, A. (2000). Biological function made crystal clear—annotation of hypothetical proteins via structural genomics. Current Opinions in Biotechnology, 11(1):25-30.
[Garey and Johnson, 1979] Garey, M. and Johnson, D. (1979). Computers and Intractability: A Guide to the theory of NP-completeness. W.H. Freeman and Co.
[Heiskanen et al., 1999] Heiskanen, T., Lundkvist, A., Soliymani, R., Koivunen, E., Vaheri, A., and Lankinen, H. (1999). Phage-displayed peptides mimicking the discontinuous neutralization sites of puumala hantavirus envelope glycoproteins. Virology, 262:321-332.
[Hoogenboom et al., 1998] Hoogenboom, H., deBruine, A., Hufton, S., Hoet, R., Arends, J., and Roovers, R. (1998). Antibody phage display technology and its applications. Immunotechnology, 4:1-20.
[Hubbell et al., 2000] Hubbell, W., Cafiso, D., and Altenbach, C. (2000). Identifying conformational changes with site-directed spin labeling. Nat Struct Biol., 7(9):735-9.
[Jameson and Wolf, 1988] Jameson, B. and Wolf, H. (1988). The antigenic index: a novel algorithm for predicting antigenic determinants. CABIOS, 4(1):181-186.
[Janeway and Travers, 1996] Janeway, C. and Travers, P. (1996). Immunobiology. Current Biology Ltd.
[Jesaitis et al., 1999] Jesaitis, A. J., Gizachew, D., Dratz, E., Siemsen, D., Stone, K., and Burritt, J. (1999). Actin surface structure revealed by antibody imprints: Evaluation of phage-display analysis of anti-actin antibodies. Protein Science, 8:760-770.
[Kabsch et al., 1990] Kabsch, W., Mannherz, H., Suck, D., Pal, E., and Holmes, K. (1990). Atomic structure of the actin:dnase I complex. Nature, 347:3744.
[Marinissen and Gutkind, 2001] Marinissen, M. and Gutkind, J. (2001). G-protein-coupled receptors and signaling networks: emerging paradigms. Trends Pharmacol. Sci., 22(7):368-76.
[McPherson, 1999] McPherson, A. (1999). Crystallization of Biological Macromolecules. Cold Springs Harbor Laboratory.
[Michel, 1990] Michel, H. (1990). Crystallization of Membrane Proteins. CRC Press.
Mumey, et al; (2003) A New Method for Mapping Discontinuous Antibody Epitopes to Reveal Structural Features of Proteins. J Comput Biol. 2003;10(3-4):555-67.
[Mills et al., 1998] Mills, J., Miettinen, H., Viases, M., and Jesaitis, A. (1998). Molecular and Cellular Basis of Inflammation, chapter The structure and function of the N-formyl peptide receptor, pages 215-245. Humana Press Inc., Totowa, N.J.
[Padlan, 1996] Padlan, E. (1996). X-ray crystallography of antibodies. Adv. Protein Chemistry, 49:57-133.
[Palczewski et al., 2000] Palczewski, K., Kumasaka, T., Hori, T., Behnke, C., Motoshima, H., Fox, B., Le, T., Teller, D., Okada, T., Stenkamp, R., Yamamoto, M., and Miyano, M. (2000). Crystal structure of rhodopsin: A g protein-coupled receptor. Science, 289:739-745.
[Pini et al., 1998] Pini, A., Viti, F., Santucci, A., Carnemolla, B., Zardi, L., Neri, P., and Neri, D. (1998). Design and use of a phage display library. human antibodies with subnanomolar affinity against a marker of angiogenesis eluted from a two-dimensional gel. J. Biol. Chem., 273:21769-21776.
[Sidhu et al., 2000] Sidhu, S., Lowman, H., Cunningham, B., and Wells, J. (2000). Phage display for selection of novel binding peptides. Methods in Enzymology, 328:333-363.
[Simons et al., 2001] Simons, K., Strauss, C., and Baker, D. (2001). Prospects for ab initio protein structural genomics. J. Mol. Biol., 306(5):1191-1199.
[Smith and Waterman, 1981] Smith, T. and Waterman, M. (1981). Identification of common molecular subsequences. J. Mol. Biol., 147:195-197.
[Teller et al., 2001] Teller, D., Okada, T., Behnke, C., Palczewski, K., and Stenkamp, R. (2001). Advances in determination of a high-resolution three-dimensional structure of rhodopsin, a model of g-protein-coupled receptors (gpcrs). Biochemistry, 40:7761-7772.
[Wess, 1997] Wess, J. (1997). G-protein-coupled receptors: molecular mechanisms involved in receptor activation and selectivity of g-protein recognition. FASEB J. 11 (5):346-354.
[Young et al., 2000] Young, M., Tang, N., Hempel, J., Oshiro, C., Taylor, E., Kuntz, I., Gibson, B., and Dollinger, G. (2000). High throughput protein fold identification by using experimental constraints derived from intramolecular cross-links and mass spectrometry. Proc. Natl. Acad. Sci., 97(11):5802-5806.

We claim:

1. A method of mapping surface epitopes on a target protein comprising:
a) providing a solid support comprising an antibody capable of binding to said target protein;
b) contacting said solid support with a library of random peptides under conditions where a set of probe peptides bind to said antibody;
c) eluting said set of probe peptides;
determining the amino acid sequence of the members of said set of probe peptides;
e) computationally aligning said probe peptide sequences to said target protein;
f) generating a set of best alignments for each probe peptide sequence;
g) generating a surface-neighbor probability graph from said alignments;
h) determining edge weight of said surface-neighbor probability graph;
i) constructing a at least one surface epitope of said target protein from said surface-neighbor probability graph.

2. The method according to claim 1, wherein said surface epitope comprises a discontinuous epitope.

3. The method according to claim 1, wherein said computational aligning comprises a branch and bound program.

4. The method according to claim 1, wherein said library comprises a phage display library.

5. The method according to claim 4, wherein said method further comprises enriching said phage display library.

6. The method according to claim 1, wherein said method further comprises amplifying said set of probe peptides prior to said determining of amino acid sequence.

7. A method of mapping discontinuous epitopes on a target protein comprising:
a) providing a solid support comprising an antibody capable of binding to said target protein;
b) contacting said solid support with a library of random peptides under conditions where a set of probe peptides bind to said antibody;
c) eluting said set of probe peptides;
d) determining the amino acid sequence of the members of said set of probe peptides;
e) computationally aligning the amino acid sequences of said probe peptide sequences to said target protein;
f) constructing at least one discontinuous epitope on said target protein.

8. The method according to claim 7, wherein said computational aligning comprises a branch and bound program.

9. The method according to claim 7, wherein said library comprises a phage display library.

10. The method according to claim 9, wherein said method further comprises enriching said phage display library.

11. The method according to claim 7, wherein said method further comprises amplifying said set of probe peptides prior to said determining of amino acid sequence.

12. A method of mapping discontinuous epitopes on a target protein comprising:
a) providing a solid support comprising an antibody capable of binding to said target sequence;
b) contacting said solid support with a library of random peptides under conditions where a set of probe peptides bind to said antibody;
c) eluting said set of probe peptides;
d) determining the amino acid sequence of the members of said set of probe peptides;
e) determining a consensus epitope sequence;
f) computationally aligning said consensus epitope sequence to said target protein;
g) constructing at least one discontinuous epitope on said protein.

13. The method according to claim 12, wherein said computational aligning comprises a branch and bound program.

14. The method according to claim 12, wherein said library comprises a phage display library.

15. The method according to claim 14, wherein said method further comprises enriching said phage display library.

16. The method according to claim 12, wherein said method further comprises amplifying said set of probe peptides prior to said determining of amino acid sequence.

17. A method of mapping surface epitopes on a target protein comprising:
a) providing a solid support comprising an aptamer capable of binding to said target protein;
b) contacting said solid support with a library of random peptides under conditions where a set of probe peptides bind to said antibody;
c) eluting said set of probe peptides;
determining the amino acid sequence of the members of said set of probe peptides;
e) computationally aligning said probe peptide sequences to said target protein;
f) generating a set of best alignments for each probe peptide sequence;
g) generating a surface-neighbor probability graph from said alignments;
h) determining edge weight of said surface-neighbor probability graph;
i) constructing at least one surface epitope of said target protein from said surface-neighbor probability graph.

18. A method of mapping discontinuous epitopes on a target protein comprising:
a) providing a solid support comprising an aptamer capable of binding to said target sequence;
b) contacting said solid support with a library of random peptides under conditions where a set of probe peptides bind to said antibody;
c) eluting said set of probe peptides;
d) determining the amino acid sequence of the members of said set of probe peptides;
e) determining a consensus epitope sequence;
f) computationally aligning said consensus epitope sequence to said target protein;
g) constructing at least one discontinuous epitope on said protein.

19. A method of mapping protein-protein binding sites comprising:
a) providing a solid support comprising a first protein;
contacting said solid support with a second protein under conditions where said first protein binds to said second protein;
c) contacting said solid support with a library of random peptides under conditions where a set of probe peptides compete with said second protein for binding to said first protein;
d) eluting said set of probe peptides;
e) determining the amino acid sequences of the members of said set of probe peptides;
f) computationally aligning said amino acid sequences of the members of said set of probe peptides to said target protein;
g) constructing the binding site on said protein.


SEQUENCE LISTING (7 sequences)

SEQ ID NO: 1 - protein, 8 aa, Artificial
  Antibody Epitope
VPHPTWMR

SEQ ID NO: 2 - protein, 372 aa, Artificial
  Antibody Epitope
DEDETTALVCDNGSGLVKAXXXXXXXXXXXXXXXXXXXXXXXXXXXXXXXXXXXXXXXXXXXXXXXXXXXXXXXXXXXXXXXXXXXXXXXYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXAPPERKYSVWIGGSILASLSTFQQMWITKQEYDEAGPSIVHR

SEQ ID NO: 3 - protein, 8 aa, Artificial
  Antibody Epitope
EQQVSTAQ

SEQ ID NO: 4 - protein, 9 aa, Artificial
  Antibody Epitope
EQQASATTQ

SEQ ID NO: 5 - protein, 10 aa, Artificial
  Stability Sequence
MGXXXXGGPP

SEQ ID NO: 6 - protein, 5 aa, Artificial
  linker
GSGGS

SEQ ID NO: 7 - protein, 4 aa, Artificial
  linker
GGGS